(19) United States
(12) Patent Application Publication
(10) Pub. No.: US 2020/0047096 A1
(43) Pub. Date: Feb. 13, 2020

(54) Filtering Device, Capturing Device, and Uses Thereof

(71) Applicants: Hitachi Chemical Co., Ltd., Tokyo (JP); Hitachi Chemical Co. America, Ltd., San Jose, CA (US)

(72) Inventor: Mieko Ogura, San Clemente, CA (US)

(73) Assignees: Hitachi Chemical Co., Ltd., Tokyo (JP); Hitachi Chemical Co. America, Ltd., San Jose, CA (US)

(21) Appl. No.: 16/492,675

(22) PCT Filed: Mar. 9, 2018
(86) PCT No.: PCT/US2018/021884

§ 371 Date: Sep. 10, 2019

Related U.S. Application Data
(60) Provisional application No. 62/470,115, filed on Mar. 10, 2017.
(60) Provisional application No. 62/508,162, filed on May 18, 2017.
(60) Provisional application No. 62/569,537, filed on Oct. 7, 2017.

(51) Int. Cl. B01D 39/20; B01D 36/04; B01D 29/52; B01D 29/23; B32B 5/02; B32B 5/26; C12N 15/10

(52) U.S. Cl. CPC B01D 39/2017; B01D 36/045; B01D 29/52; B01D 29/23; B32B 5/02; B32B 2307/726; C12N 15/1003; B01D 2239/065; B01D 2239/1258; B32B 2250/20; B32B 2262/101; B32B 5/26

(57) ABSTRACT

The disclosure relates to filtering devices, capture devices and their uses to isolate nucleic acids from exosome and/or vesicles.

### BACKGROUND

The disclosure relates to filtering devices, capture devices and their uses to isolate nucleic acids from exosome and/or vesicles.

### SUMMARY

The disclosure relates to a filtering device comprising one or more wells, each of which comprising a filter and a discharge port.

The disclosure also relates to a capture device comprising one or more capture wells, wherein each of the plurality of the capture wells comprise high density polyethylene that is treated with plasma.

The disclosure further relates to a filtering system comprising the filtering device and the capture device.

The disclosure also relates to a method of isolating nucleic acids from exosome and/or vesicle in a biological sample, comprising: loading the biological sample into at least one well of a multi-well insert comprising one or more wells, each of which comprising a filter and a discharge port; passing at least a part of the biological sample through the filter to capture the exosome and/or vesicle in the filter; lysing the exosome and/or vesicle; and isolating the nucleic acids from the exosome and/or vesicle.

### BRIEF DESCRIPTION OF DRAWINGS

FIG. 1 depicts an exemplary filter device showing dimensions in inches and millimeters.

FIG. 2 depicts an exemplary capture device showing dimensions in inches and millimeters.

FIGS. 3 and 4 depict experimental exosome mRNA analysis using different exemplary filters.

### DETAILED DESCRIPTION

In one aspect, the disclosure relates to a filtering device comprising one or more wells, each of which comprising a filter and a discharge port. The “well” is a longitudinal hole defined by wall lining. In some embodiments, the well may be a tubular, spherical, or conical hole.

In some embodiments, the filter comprises first and second parts, which may be first and second layers. The first part may have a different retention rate from the second part. A layer may contain fixed boundaries distinguishing itself from another layer, but a part may not contain such fixed boundaries and may include any part of the filter. In some embodiments, the filter comprises first, second and third parts or layers. The different parts, such as the first, second, and third parts, may not overlap. In some embodiments, the retention rate in the parts or layers in upstream may be greater than the retention rate in the parts or layers in downstream. Herein, the filtrate contacts the parts or layers in the upstream before contacting the parts of layers in the downstream. In additional embodiments, at least one, two or all of the first, second and third parts or layers comprise at least one glass fiber. In further embodiments, the glass fiber is borosilicate glass fibers. In yet further embodiments, the first part or layer comprises a first glass fiber, the second part or layer comprises a second glass fiber, and the first and second glass fibers are different. In yet additional embodiments, the first, second, and third parts or layers comprise first, second, and third glass fibers, respectively. The first, second and third glass fibers may be the same or different.

In some embodiments, the first part or layer is above the second part or layer and thus contacts with the biological sample first before the second part or layer. In additional embodiments, the first part or layer is directly above the second part or layer, being connected to the second part or layer. In further embodiments, the second part or layer is above the third part or layer. In yet additional embodiments, the first layer has a thickness of at least about 0.02, 0.03, 0.04, 0.05, 0.06, 0.07, 0.08, 0.09, 0.1, 0.2, 0.3, 0.4, 0.5, 0.6, 0.7, 0.8, 0.9, or 1.0 mm. The first layer may have a thickness of about 4.0, 3.5, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1 mm or less. In additional embodiments, the first layer has a thickness of about 0.01-4.0 (from about 0.01 to about 4.0) mm, 0.02-3.0 mm, 0.1-1.0 mm, 0.2-0.3 mm, 0.2-0.4 mm, 0.1-3.0 mm, 0.25-0.30 mm, or 0.1-0.7 mm. In yet further embodiments, the second layer has a thickness of at least about 0.02, 0.03, 0.04, 0.05, 0.06, 0.07, 0.08, 0.09, 0.1, 0.2, 0.3, 0.4, 0.5, 0.6, 0.7, 0.8, 0.9, or 1.0 mm. The second layer may have a thickness of about 4.0, 3.5, 3.0, 2.5, 2.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1 mm or less. In additional embodiments, the second layer has a thickness of about 0.1-4.0 (from about 0.1 to about 4.0) mm, 0.1-3.0 mm, 0.1-2.0 mm, 0.1-1.0 mm, 0.2-0.3 mm, 0.2-4.0 mm, 0.2-3.0 mm, 0.2-2.0 mm, 0.2-1.5 mm, or 0.1-0.7 mm. In some embodiments, the third layer has a thickness of at least about 0.02, 0.03, 0.04, 0.05, 0.06, 0.07, 0.08, 0.09, 0.1, 0.2, 0.3, 0.4, 0.5, 0.6, 0.7, 0.8, 0.9, or 1.0 mm. The third layer may have a thickness of about 4.0, 3.5, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1 mm or less. In additional embodiments, the third layer has a thickness of about 0.1-4.0 (from about 0.1 to about 4.0) mm, 0.1-3.0 mm, 0.1-2.0 mm, 0.1-1.0 mm, 0.2-0.3 mm, 0.2-4.0 mm, 0.2-3.0 mm, 0.2-2.0 mm, 0.2-1.5 mm, or 0.1-0.7 mm.

In some embodiments, the retention rate of the filter is greater than 50%, 75%, 90% or 99% for vesicles having a diameter of from about 0.6 microns to about 1.5 microns in diameter. In one embodiment, the filter material captures vesicles sized from about 0.7 microns to about 1.6 microns in diameter. In one embodiment, the filter material captures exosomes or other vesicles ranging in size from about 0.020 to about 1.0 microns. The retention rate may depend on a particle retention.

In some embodiments, the first part or layer has a particle retention of at least about 0.1, 0.2, 0.3, 0.4, 0.5, 0.6, 0.7, 0.8, 0.9, 1.0, 1.1, 1.2, 1.3 or 1.4 μm. The first part or layer may have a particle retention of at least about 5.0, 4.0, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1 μm or less. In additional embodiments, the first part or layer has a particle retention of about 0.1-6.0 (from about 0.1 to about 4.0) μm, 0.4-3.0 μm, 0.2-2.0 μm, 0.2-1.5 μm, 0.5-4.0 μm, 0.4-3.0 μm, 0.5-2.0 μm, 0.6-2.0, or 1.0-2.0 μm. In yet further embodiments, the second part or layer has a particle retention of at least about 0.02, 0.03, 0.04, 0.05, 0.06, 0.07, 0.08, 0.09, 0.1, 0.2, 0.3, 0.4, 0.5, 0.6, 0.7, 0.8, 0.9, or 1.0 μm. The second part or layer may have a particle retention of about 5.0 4.0, 3.5, 3.0, 2.5, 2.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1 μm or less. In additional embodiments, the second part or layer has a particle retention of about 0.1-4.0 (from about 0.1 to about 4.0) μm, 0.4-3.0 μm, 0.2-2.0 μm, 0.4-1.0 μm, 0.4-1.5 μm, 0.2-2.0 μm, 0.2-3.0 μm, 0.2-1.0 μm, 0.5-1.0 μm, or 0.1-1.0 μm. The first part or layer may have a different particle retention from the second part or layer.

In additional embodiments, filtering device may have a third part or layer. The third part or layer may be below the second part or layer. the second part or layer has a particle retention of at least about 0.02, 0.03, 0.04, 0.05, 0.06, 0.07, 0.08, 0.09, 0.1, 0.2, 0.3, 0.4, 0.5, 0.6, 0.7, 0.8, 0.9, or 1.0 μm. The third part or layer may have a particle retention of about 5.0 4.0, 3.5, 3.0, 2.5, 2.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1 μm or less. In additional embodiments, the third has a particle retention of about 0.1-4.0 (from about 0.1 to about 4.0) μm, 0.4-3.0 μm, 0.2-2.0 μm, 0.4-1.0 μm, 0.4-1.5 μm, 0.2-2.0 μm, 0.2-3.0 μm, 0.2-1.0 μm, 0.5-1.0 μm, or 0.1-1.0 μm.

In additional embodiments, the filter may have a total volume (for example, area*thickness) of about 5, 6, 7, 8, 9, 10, 11, 12, 13, 14, 15, 16, 17, 18, 19, 20, 21, 22, 23, 24, 25 mm³ or more. The filter may have the total volume (for example, area*thickness) of about 100, 90, 80, 70, 60, 50, 40, 35, 30, 29, 28, 27, 26 mm³ or less. In additional embodiments, the filter has the total volume of about 5-100 (from about 5 to about 50) mm³, 10-50 mm³, 15-40 mm³, or 15-30 mm³.

In some embodiments, the filtering device may further comprise a pre-filter on the upstream surface of the filter described herein. The pre-filter may be effective to fix the filter. In some embodiments, the pre-filter comprises a porous polyolefin. The porous polyolefin may be a porous polyethylene.

In additional embodiments, the pre-filter has a thickness of about 0.2, 0.3, 0.4, 0.5, 0.6, 0.7, 0.8, 0.9, 1.0, 1.1, 1.2, 1.3, 1.4, or 1.5 mm or more. The pre-filter may have a thickness of about 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1 mm or less. In additional embodiments, the pre-filter has a thickness of about 0.2-5.0 (from about 0.2 to about 5.0) mm, 0.5-4.0 mm, 0.8-3.0 mm, 1.0-2.0 mm, or 1.2-1.7 mm.

In some embodiments, a pore size of the porous polyolefin has about 1, 2, 3, 4, 5, 6, 7, 8, 9, 10, 11, 12, 13, 14, or 15 μm or more. The pore size of the porous polyolefin has about 100, 90, 80, 70, 60, 50, or 40 μm or less.

In some embodiments, the outlet opening of the discharging port has at least one diameter of about 0.25, 0.30, 0.35, 0.40, 0.45, 0.50, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, or 2.30 mm or less. In additional embodiments, the outlet opening of the discharging port has at least one diameter of more than about 0.01, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.05, or 1.10 mm. In further embodiments, the outlet opening of the discharging port has at least one diameter of about 0.01-0.61 mm, 0.10-2.5 (from about 0.1 to about 2.5) mm, 0.20-2.3 mm, 0.45-0.70 mm, 0.40-2.0 mm, 0.45-1.15 mm, 0.40-1.10 mm, 0.01-0.70 mm, 0.30-0.70 mm, or 0.50-0.70 mm.

In additional embodiments, the filter may be placed in the upstream of the discharge port of the well, for example, in contact with the discharge port.

Lysis buffer may be placed into the one or more wells and lyses exosomes. The lysing reaction may require incubation time.

Lysis buffer may be remained in the filter at least 5 or 10 min at 37° C., before transferred from the filter by centrifugation. The buffer retention time during incubation may depend on the size or dimension of the outlet opening of the discharging port. The efficiency of lysing exosome may be saturated after 5 or 10 min of contacting with the lysis buffer in the filter. The efficiency of lysing exosome may be reduced less than 50% when lysis buffer is remained in contact with the exosomes for less than 5 min in the filter. The size or diameter of the outlet opening of the discharging port may determine the most effective retention time of the buffer in the filter. If the outlet opening of the discharging port is too large, the buffer may pass though too quickly, if it is too small, the buffer may be clogged.

As used herein, the term “about” modifying, for example, the quantity of an ingredient in a composition, concentrations, volumes, process temperature, process time, yields, flow rates, pressures, diameters, lengths, and like values, and ranges thereof, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities. The term “about” further may refer to a range of values that are similar to the stated reference value. In certain embodiments, the term “about” refers to a range of values that fall within 10, 9, 8,7, 6, 5,4, 3, 2, 1 percent or less of the stated reference value.

In some embodiments, the filtering device is in a form of strip having multiple wells in a row. In further embodiments, the filtering device is an eight-well filter strip. In additional embodiments, the filtering device has 1, 2, 3, 4, 5, 6, 7, 8, 9, 10, 11, 12, 13, 14, 15, 16, 17, 18, 19, 20, 24, 36, 48, 60, 72, 84, 96 or more than 96 wells. The wells may be arranged in a row and/or column.

In another aspect, the disclosure relates to a capture device comprising one or more capture wells. In some embodiments, each of the capture wells comprise high density polyethylene (HDPE). In additional embodiments, the HDPE including its surface may be treated with plasma ionized gas. Upon the plasma treatment, RNAs, including mRNA poly A tail, may be isolated using the Oligo (dT) which is immobilized on the HDPE plastic surface. For example, the carboxyl group (COO—) may be able to crosslink to 5 prime amine (NH2+) of oligo (dT)20. The HDPE may have a density of at least about 0.800, 0.850, 0.900, 0.910, 0.920, 0.930, 0.940, 0.950, or 0.960 g/cm³. The HDPE may have a density of about 1.000, 0.990, 0.980, 0.970, 0.960, 0.950 g/cm³ or less. For example, the HDPE may have a density of about 0.940-0.965 (from 0.940 g/cm³ to 0.965 g/cm³). For example, the HDPE may be Marlex 9012.

In yet additional embodiments, the capture device is in a form of strip having multiple wells in a row. In further embodiments, the capture device is an eight-well filter strip. In additional embodiments, the capture device has 2, 3, 4, 5, 6, 7, 8, 9, 10, 11, 12, 13, 14, 15, 16, 17, 18, 19, 20, 24, 36, 48, 60, 72, 84, 96 or more than 96 wells. The wells may be arranged in a row and/or column.

In another aspect, the disclosure relates to a filtering system comprising (i) the filtering device described above, and (ii) the capture device described above. The filtering device may be configured to fit in the capture device

Due to the rapid rate of nucleic acid degradation in the extracellular environment, conventional understanding suggests that many tissues are unable to provide nucleic acid that would be suitable as a diagnostic target because the nucleic acids would be degraded before they could be used as a template for detection. However, extracellular RNA (as well as other biomarkers disclosed herein) may be associated with one or more different types of membrane particles (ranging in size from 50-80 nm), exosomes (ranging in size from 50-100 nm), exosome-like vesicles (ranging in size from 20-50 nm), and micro vesicles (ranging in size from 100-1000nm). Other vesicle types may also be captured, including, but not limited to, nanovesicles, vesicles, dexosomes, blebs, prostasomes, microparticles, intralumenal vesicles, endosomal-like vesicles or exocytosed vehicles. As used herein, the terms “exosomes” and “vesicles” are used in accordance with their respective ordinary meanings in this field and shall also be read to include any shed membrane bound particle that is derived from either the plasma membrane or an internal membrane. For clarity, the terms describing various types of vesicles shall, unless expressly stated otherwise, be generally referred to as vesicles or exosomes. Exosomes may also include cell-derived structures bounded by a lipid bilayer membrane arising from both herniated evagination (e.g., blebbing) separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the exosome lumen, including but not limited to tumor-derived microRNAs or intracellular proteins. Exosomes may also include membrane fragments. Circulating tumor-derived exosomes (CTEs) as referenced herein are exosomes that are shed into circulation or bodily fluids from tumor cells. CTEs, as with cell-of-origin specific exosomes, typically have unique biomarkers that permit their isolation from bodily fluids in a highly specific manner. As achieved by several embodiments disclosed herein, selective isolation of any of such type of vesicles allows for isolation and analysis of their RNA (such as mRNA, microRNA, and siRNA) which can be useful in diagnosis or prognosis of numerous diseases. Thus, exosomes and microvesicles (EMV) can provide biomarkers for diseases (including, but not limited to, the isolation of vesicles from urine for the assessment of renal disease). Target compounds that can be extracted using the devices and methods herein disclosed include proteins, lipids, antibodies, vitamins, minerals, steroids, hormones, cholesterol, amino acids, vesicles, exosomes, and nucleic acids.

In several embodiments, biological fluid samples are processed. As used herein, a “bodily fluid” shall be given its ordinary meaning and may also refer to a sample of fluid collected from the body of the subject, including but not limited to, for example, blood, plasma, serum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof.

In another aspect, the disclosure relates to a method of isolating nucleic acids from exosome and/or vesicle in a biological sample, comprising: loading the biological sample into at least one well of a multi-well insert comprising one or more wells, each of which comprising a filter and a discharge port, wherein the filter comprises first and second parts or layers; passing at least a part of the biological sample through the filter to capture the exosome and/or vesicle in the filter; lysing the exosome and/or vesicle; and isolating the nucleic acids from the exosome and/or vesicle. In some embodiments, the multi-well insert may be the filtering device described above. In additional embodiments, the isolating comprises collecting the nucleic acids from the exosome and/or vesicle into a capture well. The capture well may comprise the HDPE as described above for the capture device. In yet additional embodiments, the isolating comprises collecting the nucleic acids from the one or more wells of the multi-well insert into one or more capture wells. In further embodiments, the one or more capture wells may be the capture device described above.

In some embodiments, the passing comprises passing the biological sample from the one or more wells of the multi-well insert into a plate comprising one or more wells. In additional embodiments, the collecting comprises centrifuging the multi-well insert. In further embodiments, the passing comprises centrifuging the multi-well insert.

In some embodiments, the biological fluid samples may include a sample selected from the group consisting of RNAs, DNA, protein, exosomes, vesicles, other circulating membrane bound nucleic acid and/or protein-containing structures, and carbohydrate. The RNAs may comprise RNA selected from the group consisting of poly(A)+RNA, mRNA, miRNA, rRNA, tRNA, and vRNA.

In some embodiments, the biological sample is selected from the group consisting of blood, serum, plasma, urine, sweat, saliva, ascites, peritoneal fluids, culture media and stool. In further embodiments, the method described herein may comprise collecting the biological sample from a subject. The subject may be human, animal or plant.

### EXAMPLE

TABLE 1
Experimental data from Examples

| | First layer | | Second layer | | Third layer | | Total | Outlet |
|---|---|---|---|---|---|---|---|---|
| | | Particle | | Particle | | Particle | volume of | opening of the |
| | Thickness | retention | Thickness | retention | Thickness | retention | the filter | discharging port |
| | (mm) | (μm) | (mm) | (μm) | (mm) | (μm) | (mm³) | (mm) |
| Example 1 | 0.28 | 1.2 | 0.44 | 0.7 | — | — | 16.07 | 0.61 |
| Example 2 | 0.44 | 0.7 | 0.44 | 0.7 | — | — | 16.07 | 0.61 |
| Example 3 | 0.28 | 1.2 | 0.44 | 0.7 | 0.44 | 0.7 | 25.88 | 0.61 |
| Example 4 | 0.28 | 1.2 | 0.28 | 1.2 | 0.44 | 0.7 | 22.31 | 0.61 |
| Co-example 1 | 0.28 | 1.2 | 0.44 | 0.7 | — | — | 16.07 | 1.6 |

Various volume of plasma sample (800 μL-100 μL) were applied to each device. The filters are used Ahlstrom's filter #111 (Particle retention; 1.2 μm) and #151(Particle retention; 0.7 μm). Furthermore, the pre-filters are used Porex IRM-1564 (Porex Technologies Corporation, porous polyethylene, Thickness; 1.53 mm, Pore size; 15-40 μm)

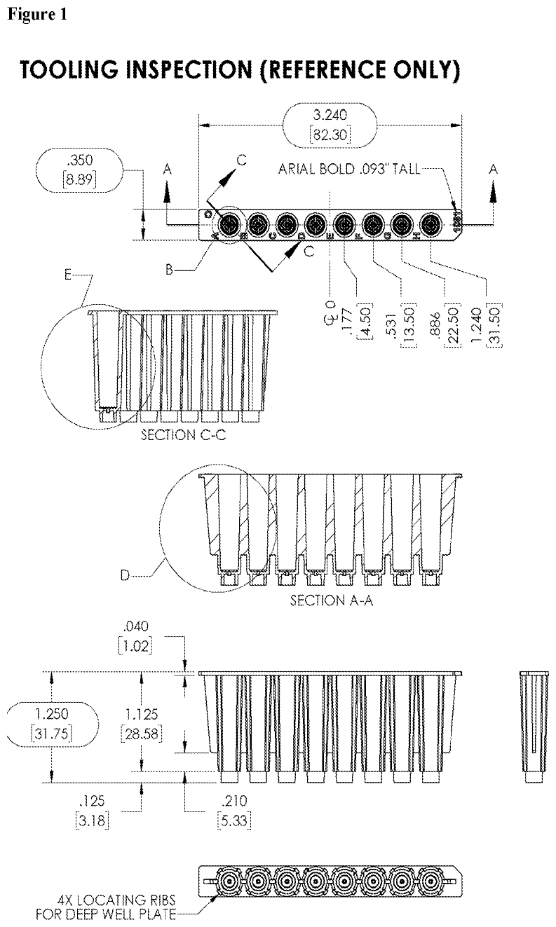

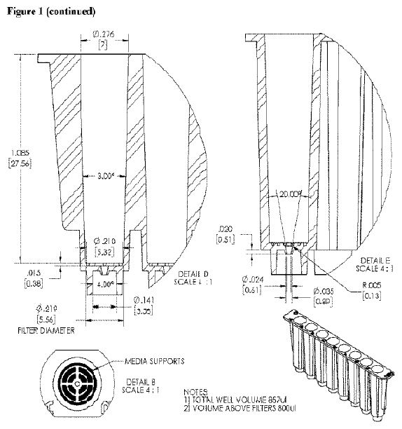

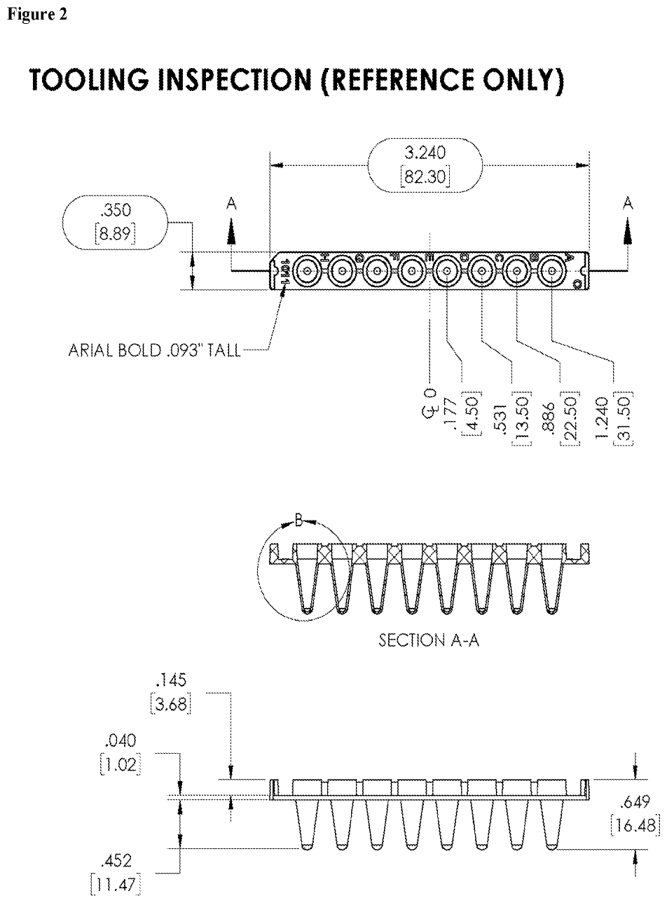

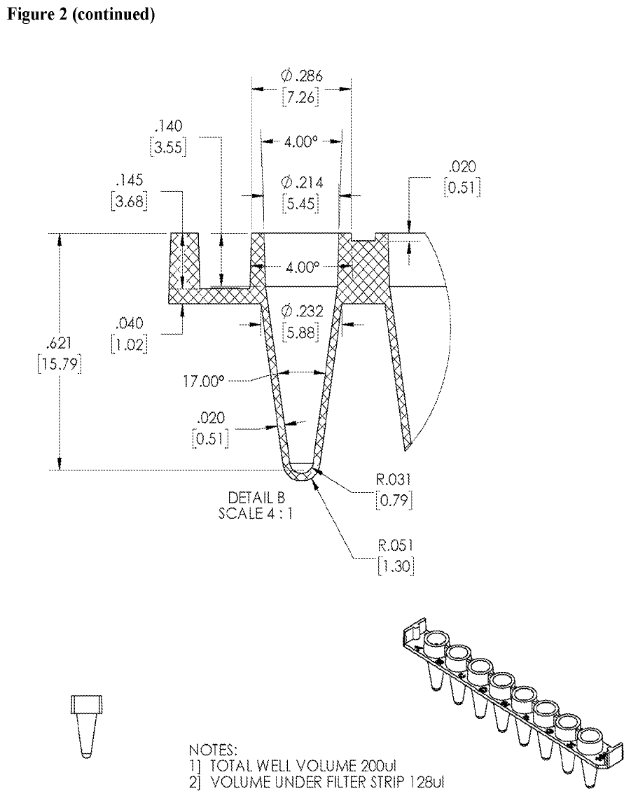

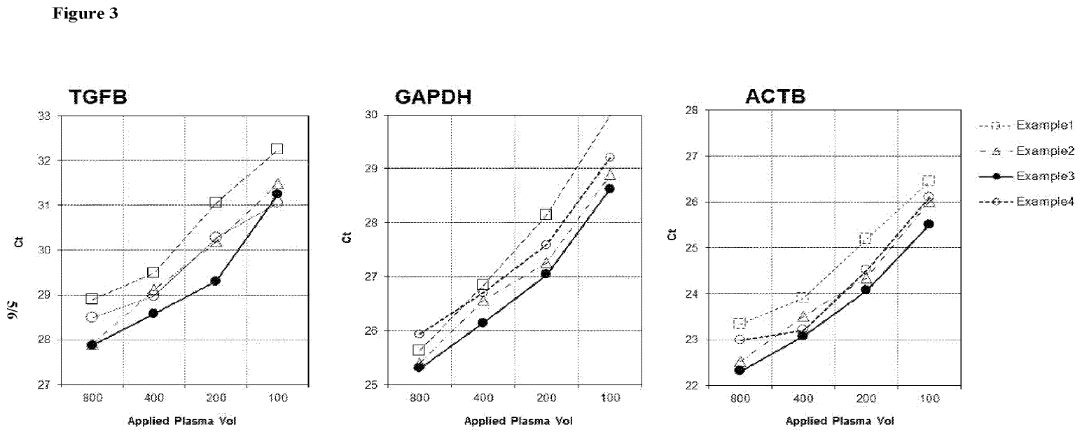

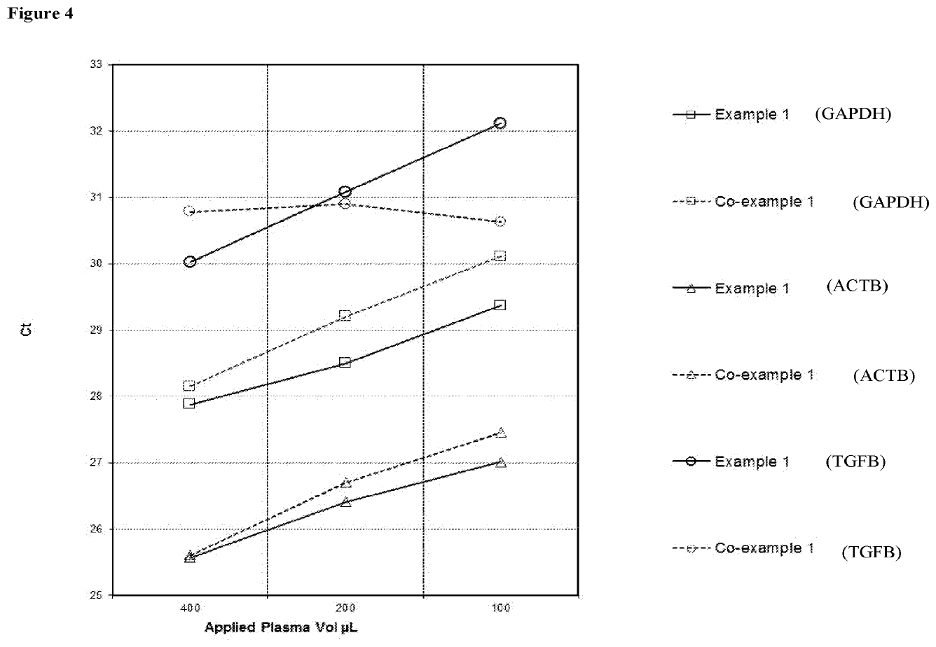

After Extracellular vesicles (EVs) were captured on the filter membrane, EVs were lysed by adding lysis buffer and transferred to oligo (dT) immobilized microtiter plate for Poly(A)+mRNA hybridization. After mRNA hybridization, cDNA was synthesized in the well directly with random hexamers and specific mRNA Transforming growth factor beta (TGF-β), Glyceraldehyde 3-phosphate dehydrogenase (GAPDH) and Beta-actin (ACTB) were amplified with real-time qPCR instrument (ViiA 7, Thermo Fisher-ABI) by using the SYBR green chemistry. A low threshold cycle (Ct) value means being better in capture rate for the Exosome. As shown in FIG. 1, Example 3 obtained the best performance over other Example.

Various volume (400 μL,200 μL and 100 μL) of plasma sample was applied to EV plate (400 μL/well) with Example 1 and Co-example 1 in device. EVs were captured on the filter membrane by centrifugation. Captured EVs were lysed by adding lysis buffer and transferred to oligo (dT) immobilized microtiter plate for Poly(A)+mRNA hybridization. After mRNA hybridization, cDNA was synthesized in the well directly with random hexamers and specific mRNA Transforming growth factor beta (TGF-β), Glyceraldehyde 3-phosphate dehydrogenase (GAPDH) and Beta-actin (ACTB) were amplified with real-time qPCR instrument (ViiA 7, Thermo Fisher-ABI) by using the SYBR green chemistry. With a large outlet opening (Co-example 1), lysis buffer passed through the filter layers, which resulted in underperformance over the Example 1. Additional experiments using the same filters but outlet openings with different diameters are performed, and the results are shown in Table 2 below.

TABLE 2
Experimental data using different diameter of
the outlet opening of the discharging port

| | Outlet opening of the | |
|---|---|---|
| | discharging port (mm) | Retained % |
| Example 5 | 0.61 | 100% |
| Example 6 | 0.9 | 70% |
| Example 7 | 1.0 | 61% |
| Example 8 | 1.19 | 51% |
| Co-example 2 | 1.6 | 35% |

Colored Lysis Buffer (60μL) was applied to each well (n=3) having the above filter strips and immediately started timer. Strips are placed on the deep well plate, sealed the top and placed in the 37° C. incubator for 5 min. After 5 min incubation, the retention of lysis buffer was measured in each well.

1-18. (canceled)

19. A method of isolating nucleic acids from exosome and/or vesicle in a biological sample, comprising:
loading the biological sample into at least one well of a multi-well insert comprising one or more wells, each of which comprising a filter and a discharge port, wherein the filter comprises first and second parts;
passing at least a part of the biological sample through the filter to capture the exosome and/or vesicle in the filter;
lysing the exosome and/or vesicle; and
isolating the nucleic acids from the exosome and/or vesicle.

20. The method according to claim 19, wherein an outlet opening of the discharging port has a diameter of less than 1.5 mm.

21. The method according to claim 19, wherein an outlet opening of the discharging port has a diameter from 0.45 mm to 1.15 mm.

22. The method according to claim 19, wherein an outlet opening of the discharging port has a diameter from 0.45 mm to 0.70 mm.

23. The method according to claim 19, wherein the first and second parts comprise glass fibers.

24. The method according to claim 19, wherein the first and second parts comprise borosilicate glass fibers.

25. The method according to claim 19, wherein the first part forms a first layer, and the second part forms a second layer.

26. The method according to claim 25, wherein the first layer has a thickness from 0.1 mm to 1 mm, and the second layer has a thickness from 0.2 mm to 1.5 mm.

27. The method according to claim 19, wherein the first part comprises a first glass fiber, the second part comprises a second glass fiber, and the first and second glass fibers are different.

28. The method according to claim 19, wherein the first part has particle retention rate from 0.6 μm to 2.0 μm, and the second part has particle retention rate from 0.4 μm to 1.5 μm.

29. The method according to claim 19, wherein the isolating comprises collecting the nucleic acids from the exosome and/or vesicle into a capture well.

30. The method according to claim 29, wherein the capture well comprises high density polyethylene having a density from 0.940 g/cm³ to 0.965 g/cm³.

31. The method according to claim 29, wherein the isolating comprises collecting the nucleic acids from the one or more wells of the multi-well insert into one or more capture wells.

32. (canceled)

33. The method according to claim 29, wherein the collecting comprises centrifuging the multi-well insert.

34. (canceled)

35. The method according to claim 29, wherein the passing comprises passing the biological sample from the one or more wells of the multi-well insert into a plate comprising one or more wells.

36. The method according to claim 29, wherein the nucleic acids are DNAs.

37. The method according to claim 29, wherein the nucleic acids are RNAs.

38. The method according to claim 37, wherein the RNAs comprises RNA selected from the group consisting of poly(A)+RNA.

39. The method according to claim 29, wherein the biological sample is selected from the group consisting of blood, serum, plasma, urine, sweat, saliva, ascites, peritoneal fluids, culture media and stool.

40. The method according to claim 29, wherein the passing comprises centrifuging the multi-well insert.
